(19) United States
(12) Patent Application Publication
(10) Pub. No.: US 2004/0005550 A1
(43) Pub. Date: Jan. 8, 2004

(54) Methods and compositions useful for targeting activated vitronectin receptor alphavbeta3

(75) Inventors: Sanford J. Shattil, La Jolla, CA (US); Glen R. Nemerow, Encinitas, CA (US); Takaaki Hato, Imabari (JP); Dwayne G. Stupack, San Diego, CA (US); Nisar A. Pampori, Frederick, MD (US)

Correspondence Address: THE SCRIPPS RESEARCH INSTITUTE, OFFICE OF PATENT COUNSEL, TPC-8, 10550 NORTH TORREY PINES ROAD, LA JOLLA, CA, 92037, US

(73) Assignee: The Scripps Research Institute, La Jolla, CA (US)

(21) Appl. No.: 10/454,660

(22) Filed: Jun. 3, 2003

Related U.S. Application Data
(63) Continuation of application No. 09/454,925, filed on Dec. 3, 1999, now abandoned.
(60) Provisional application No. 60/145,458, filed on Jul. 23, 1999.
(60) Provisional application No. 60/110,950, filed on Dec. 4, 1998.

(51) Int. Cl. C12Q 1/70; G01N 33/53; G01N 33/567; G01N 33/574; A61K 39/395; A61K 48/00

(52) U.S. Cl. 435/5; 435/7.2; 424/143.1; 435/7.23; 424/93.2

(57) ABSTRACT

The present invention provides ligands which can selectively bind to activated αᵥβ₃ integrin. A novel monovalent ligand-mimetic (WOW-1 Fab) which includes a single αᵥ integrin-binding domain from multivalent adenovirus penton base is provided. Further, the present invention describes particular compositions of activated αᵥβ₃-specific ligands, such as an antibody which immunoreacts preferentially with activated αᵥβ₃ integrin. The invention also describes methods using an activated αᵥβ₃-specific ligand for diagnostic detection of activated αᵥβ₃ integrin in tissues and for the targeted delivery of therapeutic agents to tissues containing activated αᵥβ₃ integrin.

### TECHNICAL FIELD

The invention relates to ligands which bind to activated vitronectin receptor αᵥβ₃. The invention also relates to methods using these ligands for diagnostic detection of activated αᵥβ₃ and for targeted delivery of therapeutic agents to activated αᵥβ₃ and to tissues containing activated αᵥβ₃.

### BACKGROUND OF THE INVENTION

The integrin known as the vitronectin receptor αᵥβ₃ is well characterized and known to play a role in a variety of biological processes including proliferation of endothelial cells, osteoclasts and arterial smooth muscle cells. Further, it is involved in the biological processes of angiogenesis, arterial restenosis, bone remodeling, osteoporosis and tumor progression. It is further known in the art that integrins mediate cell adhesion and signaling during many developmental, physiological and pathological processes. However, the role of activation of αᵥβ₃ in biological processes is not well understood at present. The β₃ integrin family includes α_{IIb}β₃, often referred to as the fibrinogen receptor, and αᵥβ₃, the vitronectin receptor. α_{IIb}β₃ is confined to megakaryocytes and platelets and is required for platelet aggregation through interactions with Arg-Gly-Asp (RGD)-containing adhesive ligands, including fibrinogen and von Willebrand factor. The vitronectin receptor (αᵥβ₃ integrin) is more widely expressed in proliferating endothelial cells, arterial smooth muscle cells, osteoclasts, platelets and certain subpopulations of leukocytes and tumor cells. The list of cognate ligands for αᵥβ₃ overlaps that of α_{IIb}β₃ but includes others, such as osteopontin, matrix metalloproteinase-2, and adenovirus penton base, which do not interact with the fibrinogen receptor α_{IIb}β_{3.}

One fundamental function of integrins is ligand binding, which in many cases is rapidly regulated by a process variously referred to as “integrin activation”, “inside-out signaling” or “affinity/avidity modulation”. Integrin activation encompasses at least two events: 1) modulation of receptor affinity through conformational changes in the αβ heterodimer; and 2) modulation of receptor avidity through facilitation of lateral diffusion and/or clustering of heterodimers. Studies of α_{IIb}β₃ activation have been facilitated by the use of soluble ligands, most notably a multivalent, ligand-mimetic antibody called PAC1, and its monovalent Fab fragment, which contain an RG/YD tract in H-CDR3 (complementarity determining region no. 3 of the heavy chain) (Shattil, S. J., Kashiwagi, H., and Pampori, N. (1998) Blood 91, 2645-2657; Abrams, C., Deng, J., Steiner, B., and Sihattil, S. J. (1994) J. Biol. Chem. 269, 18781-18788). The significance of inside-out signaling, and in particular affinity modulation, for αᵥβ₃ has been less certain. The ligand binding function of αᵥβ₃ has usually been assessed by cell adhesion assays, and these have clearly shown that activation of certain cells leads to αᵥβ₃-mediated adhesion. However, adhesion assays can be strongly influenced by post-ligand binding events, including changes in cell shape, that can obscure the precise contributions of affinity or avidity modulation to the overall response.

In summary, it is known in the art that αᵥβ₃ integrin mediates diverse responses in vascular cells, ranging from cell adhesion, migration and proliferation to uptake of adenoviruses. However, the extent to which αᵥβ₃ is regulated by changes in receptor conformation (affinity), receptor diffusion/clustering (avidity) or post-receptor events is unknown.

### SUMMARY OF THE INVENTION

The present invention provides ligands which can selectively bind to activated αᵥβ₃ integrin. A novel monovalent ligand-mimetic (WOW-1 Fab) was created by replacing the H-CDR3 of PAC1 Fab with a single αᵥ integrin-binding domain from multivalent adenovirus penton base. The WOW-1 Fab and adenoviral penton base protein were used to determine the role of affinity modulation of αᵥβ₃ integrin. Both WOW-1 Fab and penton base bound selectively to activated αᵥβ₃ but not to α_{IIb}β₃ integrin in receptor and cell binding assays. Accordingly, the present invention describes particular compositions of activated αᵥβ₃-specific ligands, such as an antibody which immunoreacts preferentially with activated αᵥβ₃ integrin. Further, the invention describes methods using an activated αᵥβ₃-specific ligand for diagnostic detection of activated αᵥβ₃ integrin in tissues and for the targeted delivery of therapeutic agents to tissues containing activated αᵥβ₃ integrin.

### BRIEF DESCRIPTION OF THE FIGURES

FIG. 1. Binding of Soluble Alexa-Penton Base and WOW-1 Fab to CHO Cells Expressing αᵥβ₃.

In panel A, αᵥβ₃-CHO cells or parental CHO cells were incubated with primary antibodies specific for αᵥβ₃ (LM609), α_{IIb}β₃ (D57) or αᵥβ₅ (P1F6), and antibody binding was detected with FITC-labeled secondary antibody as described in Experimental Procedures. Cells stained with secondary antibody only were used as a negative control. For comparison, antibody binding to parental CHO cells was also studied. In panel B, the αᵥβ₃-CHO cells were incubated with either 75 nM Alexa-Penton Base (aPB) or 106 nM WOW-1 Fab for 30 min at room temperature, in the absence or presence of a 1:50 dilution of AP5 ascites to activate αᵥβ₃ or 5 mM EDTA to inhibit specific ligand binding. Then binding of aPB and WOW-1 Fab was measured by flow cytometry as described in Experimental Procedures. The data represent specific ligand binding, defined as that inhibited by EDTA, and are presented as means±SEM of three independent experiments. Similar results were obtained if αᵥβ₃ was stimulated with the purified Fab fragment of another activating antibody (LIBS6) instead of AP5 ascites. Asterisks indicate that ligand binding was significantly greater in the presence than in the absence of AP5 (P<0.01).

FIG. 2. Effect of Integrin Inhibitors on Binding of aPB and WOW-1 Fab to αᵥβ₃-CHO Cells.

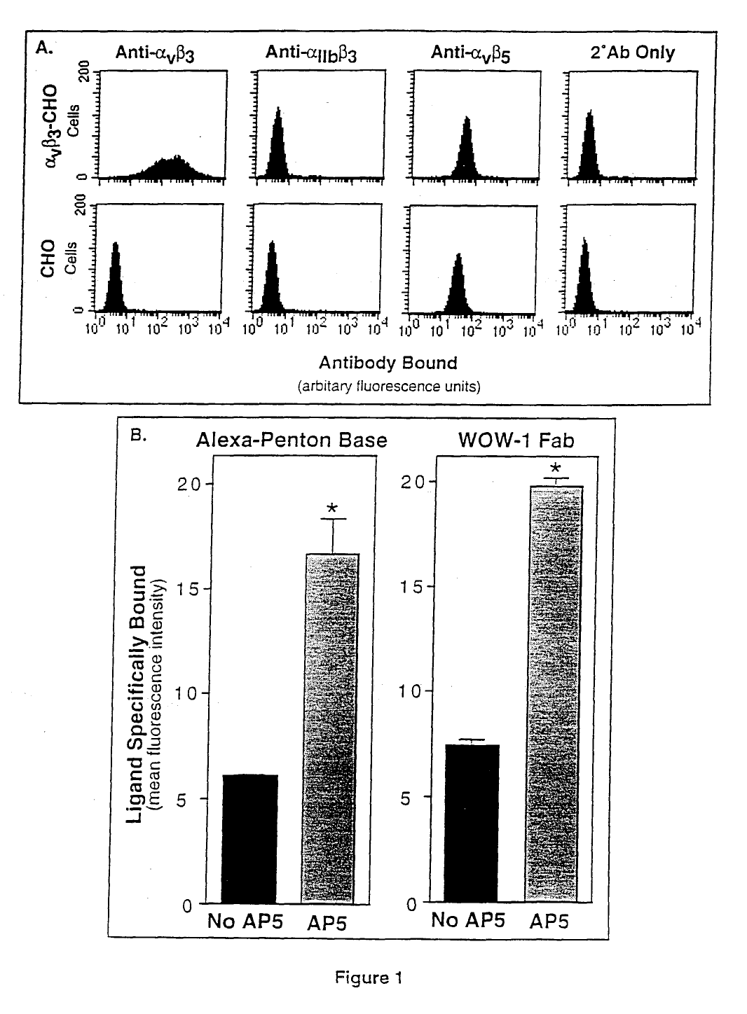

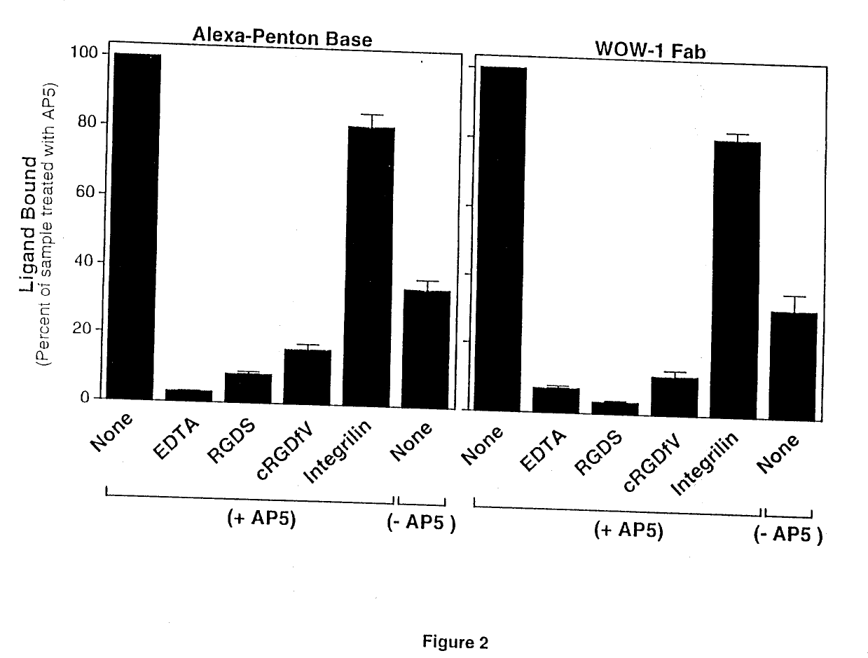

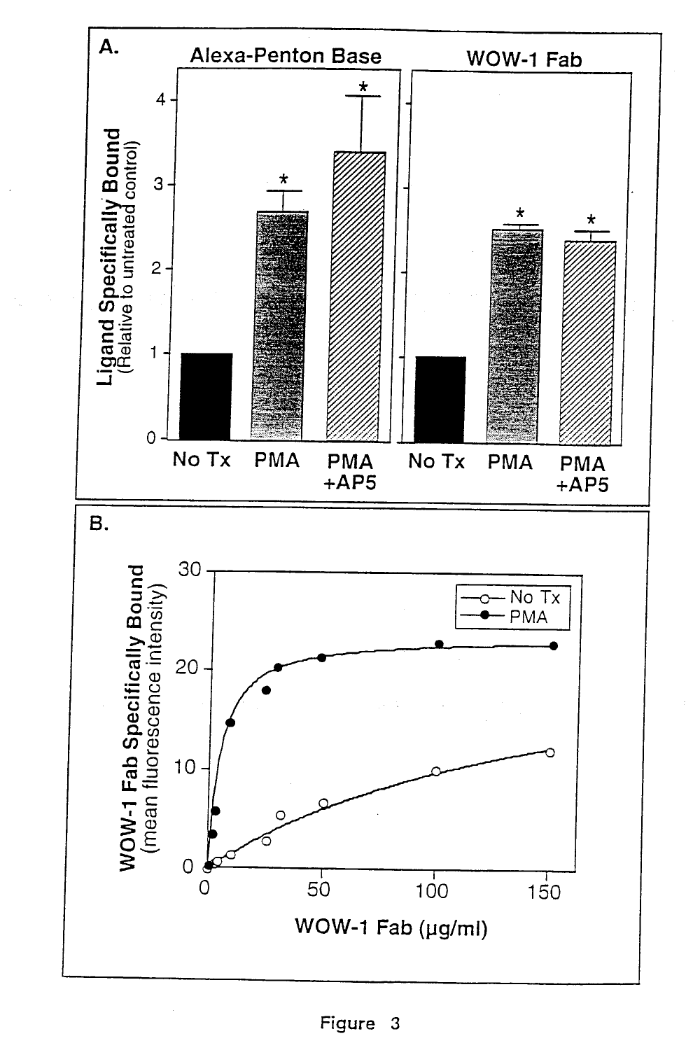

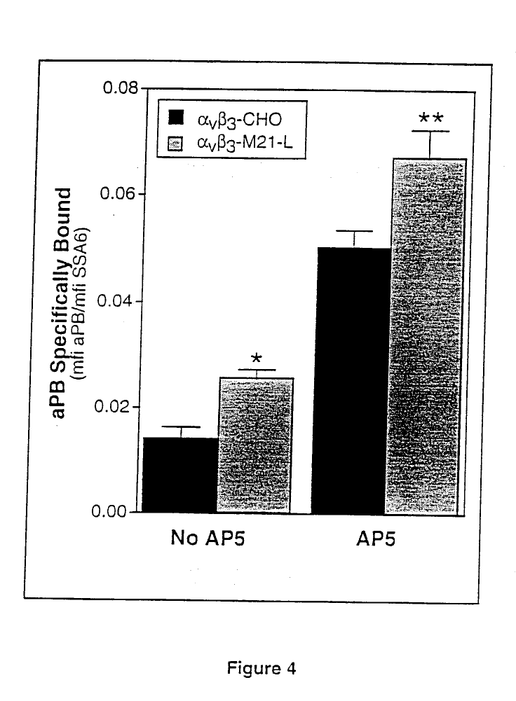

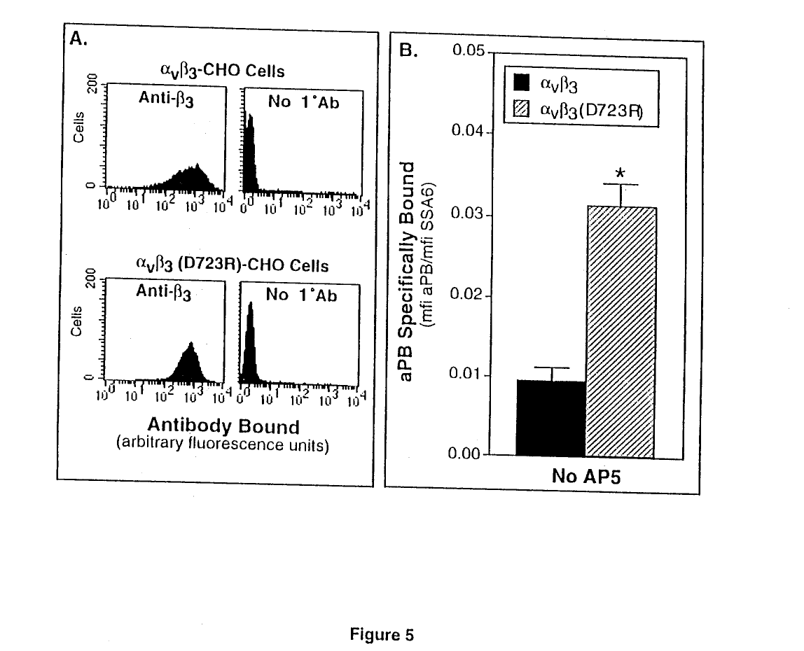

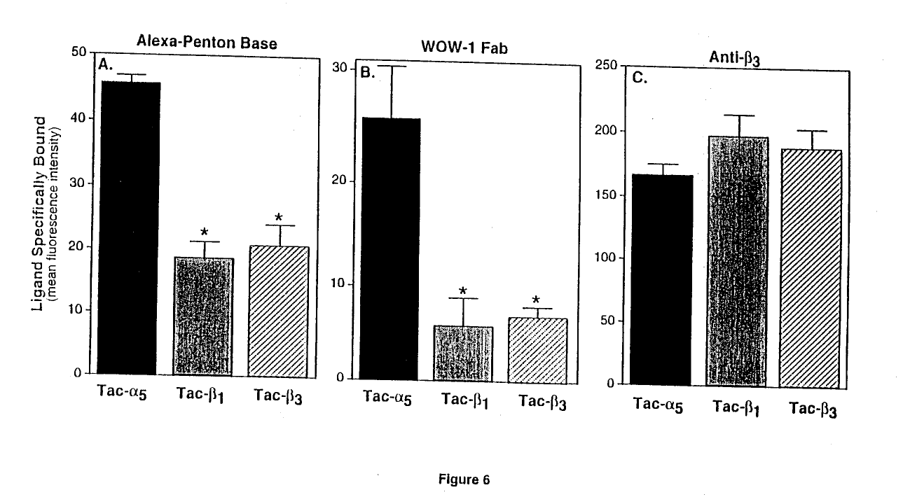

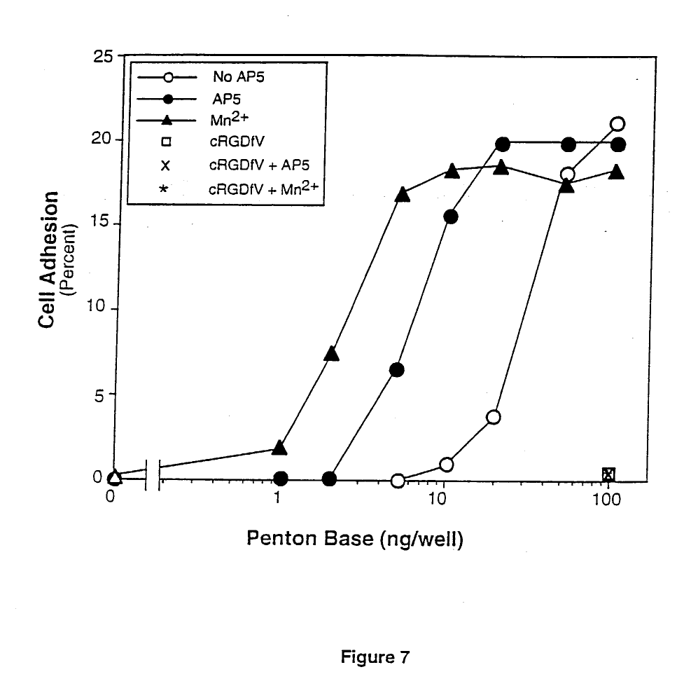

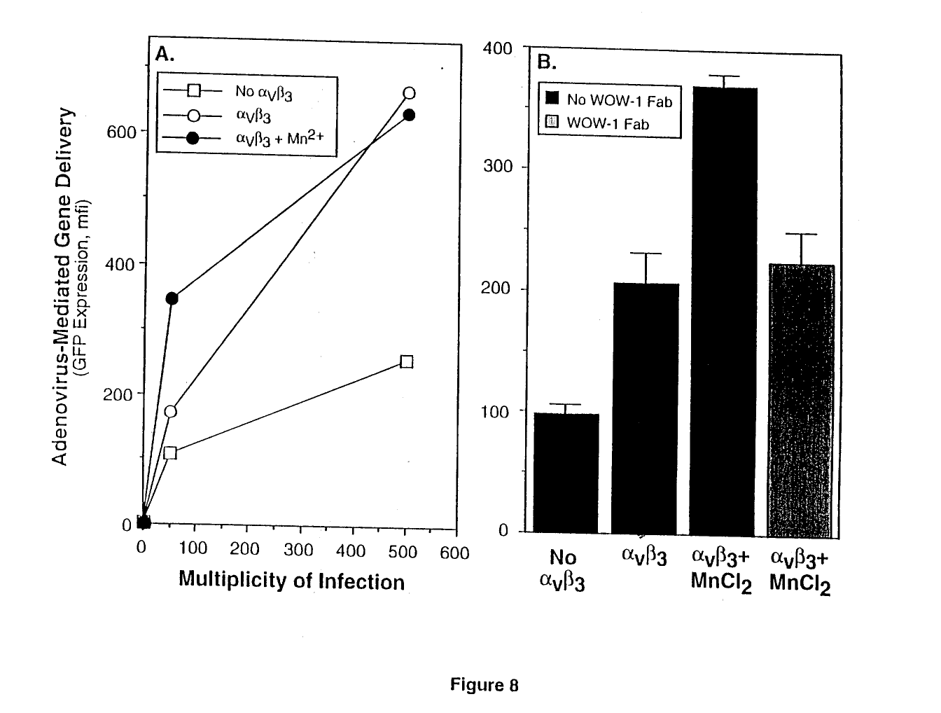

Ligand binding was carried out as in FIG. 1 in the presence of AP5 ascites (1:50) and an integrin inhibitor, as indicated. EDTA was 5 mM, RGDS 2 mM, cRGDfV 50 μM, and Integrilin 1 μM. Data are plotted as a percentage of the value for the AP5-treated sample in the absence of an inhibitor, and represent means±SEM of three experiments.

FIG. 3. αᵥβ₃ is Susceptible to Affinity Modulation by Inside-Out Signals.

In panel A, JY lymphoblastoid cells were incubated in the presence of either 75 nM aPB or 425 nM WOW-1 Fab for 15 min without an agonist (No Tx), with 100 nM phorbol myristate acetate (PMA), or with phorbol myristate acetate plus AP5 ascites (1:50). Then specific ligand binding was determined by flow cytometry. Data are the means±SEM of three experiments. Asterisks denote a significant difference compared to the No Tx sample (P<0.05). In panel B, binding of WOW-1 to JY cells was examined over a range of Fab concentrations. The data are plotted as specific (RGDS-inhibitable) binding and were subjected to non-linear regression analysis for binding to a single site. Values for apparent Kd and maximal binding are presented in Table 1. The curves are computer-generated best fits of the data. Goodness of fit (R²) values ranged from 0.94-1.00.

FIG. 4. Comparison of aPB Binding to αᵥβ₃-CHO Cells and αᵥβ₃-M21-L Melanoma Cells.

Binding of aPB (75 nM) to each cell line was carried out as described in the legend to FIG. 1. Specific aPB binding is expressed on a per receptor basis as the mean fluorescence intensity (mfi) of aPB binding divided by the mfi of SSA6 binding. Each bar represents the mean±SEM of four experiments. Single and double asterisks denote P values of <0.01 and <0.05, respectively, for the difference between the CHO cells and melanoma cells.

FIG. 5. Effect of an Activating Mutation in the β₃ Integrin Cytoplasmic Tail on the Binding of Penton Base to αᵥβ₃.

In panel A, stable CHO cell lines expressing either αᵥβ₃ or αᵥβ₃ (D723R) were stained with anti-β₃ antibody SSA6 and phycoerythrin-streptavidin to assess surface expression of αᵥβ₃. In panel B, specific binding of aPB (75 nM) was studied as described in the legend to FIG. 1. aPB binding is expressed on a per receptor basis. Data represent the means±SEM of four experiments. Asterisk denotes a difference between αᵥβ₃ and αᵥβ₃ (D723R) at the P<0.01 level. For comparison, the corresponding value for aPB binding to AP5-treated αᵥβ₃-CHO cells was 0.034±0.002.

FIG. 6. Effect of Overexpression of Isolated Integrin Cytoplasmic Tails on ligand binding to CS-1 Melanoma Cells Expressing αᵥβ₃.

As described in the Examples hereinbelow, αᵥβ₃-CS-1 cells were transiently-transfected with either the Tac-α₅, Tac-β₁ or Tac-β₃ chimera. Forty-eight hours after transfection, the cells were incubated for 30 min at room temperature with (A) 150 nM aPB or (B) 425 nM WOW-1 Fab, in the presence or absence of 5 mM EDTA. The cells were stained with anti-Tac antibody and phycoerythrin-conjugated anti-mouse IgG in order to set a live-gate on the Tac-expressing cells, and specific binding of aPB and WOW-1 Fab was measured by flow cytometry. Panel C shows that the Tac constructs had no effect on expression levels of αᵥβ₃, as monitored with anti-β₃ antibody, SSA6. Data represent the means±SEM of three experiments. The asterisks indicate that ligand binding in the presence of Tac-β₁ or Tac-β₃ was significantly less than with Tac-α₅ (P<0.01).

FIG. 7. Effect of αᵥβ₃ Activation on the Adhesion of αᵥβ₃-CHO Cells to Penton Base.

As described in the Examples hereinbelow, microtiter wells were coated with penton base and the adhesion of αᵥβ₃-CHO cells was studied for 90 min at 37° C., either with no additive (open circles), AP5 ascites (1:50; closed circles), or MnCl₂ (0.25 mM; closed triangles). Some aliquots were also incubated with 50 μM cRGDfV under each of these conditions (open square, cross, and asterisk) to assess whether cell adhesion was dependent on the presence of αᵥ integrins. This experiment is representative of three so performed.

FIG. 8. Effect of αᵥβ₃Expression and Activation on Adenovirus-Mediated Gene Delivery.

In panel A, parental CS-1 cells (No αᵥβ₃) and αᵥβ₃-CS-1 cells were incubated for 1 hour with an adenovirus vector encoding GFP at a multiplicity of infection of 50 or 500. In addition, aliquots of the αᵥβ₃-CS-1 cells were incubated with virus in the presence of 2.5 mM MnCl₂to induce maximal integrin activation. Viral infection and gene delivery were assessed 72 hours later by quantitating cellular expression of GFP by flow cytometry. Panel A depicts a single experiment, and Panel B shows the means±SEM of three experiments conducted at an m.o.i. of 50. The 4^{th} bar (from the left) of Panel B shows the effect of preincubating αᵥβ₃-CS-1 cells with 1.7 μM WOW-1 Fab for 20 min before addition of virus.

### DETAILED DESCRIPTION OF THE INVENTION

The present invention provides ligands which can selectively bind to activated αᵥβ₃ integrin. These activated αᵥβ₃-specific ligands are of particular use in the methods and compositions described in the present invention. The ability to specifically detect and interact with activated αᵥβ₃ was not available before this invention was made, and, by employing ligands of this invention, it has now been discovered that the vitronectin receptor αᵥβ₃ has an activated state under certain biological conditions, which can be useful for diagnostic and therapeutical purposes, in particular, for the targeting of therapeutical agents to certain tissues.

In order to determine the role of affinity modulation of αᵥβ₃, a novel monovalent ligand-mimetic (WOW-1) was created by replacing the H-CDR3 of PAC1 Fab with a single αᵥ integrin-binding domain from multivalent adenovirus penton base. Both WOW-1 Fab and penton base bound selectively to activated αᵥβ₃ but not to α_{IIb}β₃ integrin in receptor and cell binding assays. Accordingly, the present invention includes particular compositions of activated αᵥβ₃-specific ligands, such as an antibody which immunoreacts preferentially with activated αᵥβ₃ integrin. Further, in another embodiment the present invention describes methods using an activated αᵥβ₃-specific ligand for diagnostic detection of activated αᵥβ₃ in tissues and for targeted delivery of therapeutic agents to tissues containing activated αᵥβ₃ integrin.

One aspect of the present invention is to determine whether αᵥβ₃ is subject to affinity modulation and, if so, to explore the potential pathophysiological implications of such regulation. To accomplish this task, the binding of soluble monovalent and multivalent ligands to αᵥβ₃ in several cell types is characterized, reasoning that a monovalent ligand will be sensitive to affinity modulation and a multivalent ligand will be sensitive to both affinity and avidity modulation. Penton base, a coat protein from adenovirus type 2, is selected as a multivalent ligand because each of its five subunits contains a 50 amino acid RGD tract that mediates virus internalization through αᵥ integrins. The novel WOW-1 Fab, which is created by replacing the H-CDR3 of PAC1 Fab with a single integrin-binding domain of penton base, can be used as a monovalent ligand, because replacement of the H-CDR3 of PAC1 switches the selectivity of the Fab from activated α_{IIb}β₃ to activated αᵥβ₃ integrin, thereby enabling a direct assessment of the αᵥβ₃ affinity state. Thus, the resulting monovalent Fab, WOW-1, retains the activation-dependent characteristics of the PAC1 antibody and of the penton base protein and interacts with αᵥβ₃ integrin but not α_{IIb}β₃ integrin. Using WOW-1 Fab to study αᵥβ₃ integrin, several conclusions regarding αᵥβ₃ integrin function could be reached: The basal affinity state of αᵥβ₃ varies among cell types, being extremely low in lymphoid cells and higher in melanoma cell lines. Further, αᵥβ₃ is subject to rapid affinity modulation by inside-out signals, including those downstream of protein kinase C. At least some of the cellular signals that regulate αᵥβ₃ affinity converge at the cytoplasmic tails of the integrin. Affinity modulation has direct functional consequences, both for the adhesion and signaling functions of αᵥβ₃ and for adenovirus-mediated gene transfer. Thus, the present invention establishes that αᵥβ₃ is subject to affinity regulation, with direct implications for the anchorage-dependent functions of αᵥβ₃ and for gene delivery to cells expressing αᵥβ₃ in particular, adenovirus-mediated gene delivery.

The present invention demonstrates that αᵥβ₃ affinity varies with the cell type. Unstimulated B-lymphoblastoid cells bind WOW-1 Fab poorly (apparent Kd=2.4 μM), but acute stimulation with phorbol myristate acetate increases receptor affinity >30-fold (Kd=80 nM), with no change in receptor number. In contrast, αᵥβ₃ in melanoma cells is constitutively active, but ligand binding can be suppressed by overexpression of β₃ cytoplasmic tails. Up-regulation of αᵥβ₃ affinity has functional consequences in that it increases cell adhesion and spreading and promotes adenovirus-mediated gene transfer. The invention therefore establishes that αᵥβ₃ is subject to rapid, regulated changes in affinity that influence the biological functions of this integrin.

The invention describes in one embodiment activated αᵥβ₃-specific ligand compositions, also referred to as ligands which preferentially bind to activated αᵥβ₃. The degree of specificity can vary but typically a ligand binds preferentially when the binding constant for activated αᵥβ₃ is greater than for other targets, such as other integrins such as the platelet receptor α_{IIb}β₃, and preferably is 2 to 1000 times greater, and more preferably is 100 to 1000 times greater. Binding activities are well known in the art and can be measured by any of a variety of methods.

A preferred activated αᵥβ₃-specific ligand is an adenovirus-2 penton base protein in isolated form, fragments of penton base protein which bind activated αᵥβ₃, or an antibody which preferentially immunoreacts with activated αᵥβ₃. Penton base (PB) protein from adenovirus-2 is well known in the art and can be prepared in a variety of ways, including the methods described hereinbelow. In addition, antibodies are well known in the art and can include polyclonal or monoclonal antibodies or functional fragments thereof, such as Fab, Fv, single chain Fv (scFv), Fd and the like fragments which include the antigen binding site portion of an antibody defined by the complementarity determining regions (CDRs) as are all well known in the art.

An antibody which immunoreacts with activated αᵥβ₃ can be prepared in a variety of ways, and therefore the invention need not be so limiting. Typically an immunogen is used which contains the desired antigenic target, in this case a sample containing activated αᵥβ₃. Following immunization, the resulting antibody can be isolated using screening assays to identify the antibody which immunoreacts with the activated αᵥβ₃ integrin. A preferred antibody is the WOW-1 antibody prepared as described hereinbelow.

Specifically, an antibody which immunoreacts with activated αᵥβ₃ is prepared in the form of a Fab antibody using recombinant nucleic acid methodologies. The antibody is prepared by substituting a 50 amino acid stretch of the adenovirus-2 penton base protein into the CDR3 portion of the cloned gene encoding the PAC1 antibody. PAC1 antibody is a well characterized and well known monoclonal antibody which immunoreacts with platelet glycoprotein receptor. The modified PAC1 antibody (designated WOW-1) is then expressed in a Drosophila expression system as a fusion protein containing a His-Tag, and purified from the Drosophila culture medium using immobilized nickel chromatography.

Specifically, the WOW-1 Fab antibody is prepared as follows. Oligonucleotides PB-For (5′-ACACAGCCATATATTACTGTGCCAGAGCGGAAGAGAACTCCAACGCG; (SEQ ID NO 1) and PB-Rev (5′-ACTGAGGTTCCTTGACCCCACGCAGCGGGGGCGGCAGCTTCTGC; (SEQ ID NO 2) were used to PCR amplify sequence from adenovirus-2 DNA, representing 50 amino acids of penton base. The DNA fragment obtained is used to replace the CDR3 portion of PAC1, in the form of Fd, by an overlap PCR using Pac1-For (5′-GCGCGGGAGATCTCAGGTGCAGCTGAAGCAGTCAGGA; (SEQ ID NO 3) and

Pac1-Rev (5′-GGCGCATGACCGGTACAATCCCTGGGCACAATTTTCTTG; (SEQ ID NO 4) while adding Bgl2 and Age1 sites, respectively. The Fd DNA fragment of this grafted “WOW-1” is Bgl2/Age1 digested and cloned into a Drosophila expression vector, pMT/BiP/V5-His B (Invitrogen, Carlsbad, Calif.) containing the Drosophila metallothionine (MT) promoter and BiP secretion signal. Similarly, Pac 1-k light chain is modified by adding Nco1 and Age1 sites, using

Pac1k-For (5′-GGCGCGGGAGATCTCCATGGGATGTlCIGATGACCCAAACTCCA; (SEQ ID NO 5) and Pac1k-Rev (5′-GGCGCATGACCGGTACACTCATTCCTGTTGAAGCTCTTG; (SEQ ID NO 6), and cloned into the Nco1/Age1 sites of pMT/BiP/V5-His B vector.

Using the calcium phosphate transfection procedure, 19 μgs each of the cloned heavy and light chains of WOW-1 were cotransfected with 1 μg of selection vector, pCoHYGRO (Invitrogen, Carlsbad, Calif.), into 3 ml culture of Drosophila melanogaster, Schneider 2 (S2) cells, at 1×10⁶ cells/ml. Stable cell lines were selected in presence of hygromycin-B. Copper sulfate at 500 μM concentration is used to induce the metallothionine promoter, and the secreted WOW-1 Fab (containing a His-Tag) is purified directly from the medium using Ni—NTA column chromatography (Qiagen, Calif.). The resulting antibody, designated Fab WOW-1, preferentially immunoreacts with activated αᵥβ₃. An exemplary binding assay suitable for demonstrating the specificity of Fab WOW-1 is described hereinbelow in Example 4.

The nucleotide and amino acid residue sequence of the resulting WOW-1 Fab antibody for both the heavy and light chain is shown hereinbelow in Example 1. In one embodiment, a preferred antibody comprises the amino acid residues shown in Example 1. More preferably, an antibody is the Fab WOW-1 described in Example 1.

In another embodiment, the invention describes methods for the detection of activated αᵥβ₃ in tissues using an activation-specific αᵥβ₃ ligand according to the present invention. There are a variety of tissues and biological conditions known in the art in which αᵥβ₃ is present and plays an important biological role, therefore making detection of activated αᵥβ₃ a useful diagnostic tool. The invention need not be limited to any particular tissue or condition insofar as there will continue to be discoveries regarding the role of activated αᵥβ₃ in biological processes.

For example, processes involving αᵥβ₃ include endothelial cell growth, particularly angiogenesis, which is mediated by vitronectin receptor αᵥβ₃, and which plays a role in a variety of disease processes. By monitoring the tissue distribution of activated αᵥβ₃ during angiogenesis, one can monitor the progression of a disease, intervene in the disease, ameliorate the symptoms, and in some cases cure the disease. Thus a diagnostic process can support therapeutic treatments.

Where the growth of new blood vessels is the cause of, or contributes to, the pathology associated with a disease, detection of activated αᵥβ₃ allows collection of information vital to prognosis and treatment of the disease. Examples include rheumatoid arthritis, diabetic retinopathy, inflammatory diseases, restenosis, and the like. The growth of new blood vessels is required to support growth of a deleterious tissue, and therefore examples of additional diseases include growth of tumors where neovascularization is a continual requirement in order that the tumor grow beyond a few millimeters in thickness, and for the establishment of solid tumor metastases.

Exemplary diseases where αᵥβ₃ is involved are described in more detail in U.S. Pat. No. 5,753,230, the disclosures of which are hereby incorporated by reference.

A diagnostic method is typically practiced by

(a) admixing a ligand of this invention with a tissue containing αᵥβ₃ to form a binding reaction admixture;

(b) maintaining the admixture under conditions sufficient for the ligand to bind the αᵥβ₃ and form a ligand-αᵥβ₃ complex, including time, temperature and physiological environmental parameters consistent with a binding reaction; and

(c) determining the presence of the ligand-αᵥβ₃ complex, and thereby the presence of any activated αᵥβ₃ present in the tissue.

The method can be practiced in vitro or in vivo, as such variation in the diagnostic arts are well known. In addition, it is known that the ligand can be labeled by a variety of methods. Exemplary labels and assay methods are described in the Examples hereinbelow.

In preferred methods, an activation specific αᵥβ₃ is selected from the group consisting of adenovirus-2 penton base, fragments of penton base which bind activated αᵥβ₃, and an antibody that immunoreacts with activated αᵥβ₃. Preferably, the ligand is the Fab antibody WOW-1.

Methods For Delivery of a Therapeutic Agent

In another embodiment, the invention describes the use of an activation specific αᵥβ₃ ligand for delivery of an agent in a therapeutic composition to a tissue containing activated vitronectin receptor αᵥβ₃ for the purpose of effecting a biological modification on the tissue. The method comprises the steps of:

(a) contacting a tissue containing αᵥβ₃ with an effective amount of a therapeutic composition comprising a ligand that binds to activated αᵥβ₃, wherein the ligand is operatively linked to an agent and the agent has a therapeutic activity;

(b) maintaining said therapeutic composition in contact with the tissue under conditions sufficient for the ligand to bind to any activated αᵥβ₃ present in the tissue and thereby deliver the agent to the tissue.

The invention may be practiced in vivo or ex vivo, such that the tissue is contacted with the therapeutic composition by administering the composition to the body of a patient containing a tissue to be treated, or by presenting a tissue or organ containing the tissue to the composition in an ex vivo procedure, as are well known.

The agent can be any of a variety of materials which ultimately effects a biological response of therapeutic nature, and therefore the invention is not intended to be limited in this regard. Exemplary agents include any biologically active compound, such as a conjugated drug, toxin, biologically active peptide or protein, hormones, and the like compounds, nucleic acids such as may be active as an antisense molecule, a catalytic nucleic acid molecule, such as a ribozyme, or in gene transfer, and the like. Such methods and compositions are generally well known in the art, and therefore the invention need not be so limited.

In one embodiment, the present invention describes the use of an activation specific αᵥβ₃ ligand for gene delivery to a tissue containing activated vitronectin receptor αᵥβ_{3.} Gene delivery or gene transfer vehicles may be derived from viruses, such as, for example, adenoviruses, retroviruses, lentiviruses, adeno-associated virus, and Herpes viruses, which have a viral surface protein which has been modified to include an activation specific αᵥβ₃ ligand. Alternatively, the gene delivery or gene transfer vehicle may be a non-viral gene delivery or gene transfer vehicle, such as a plasmid, to which is bound an activation specific αᵥβ₃ ligand. In another example, the gene delivery or gene transfer vehicle may be a proteoliposome which encapsulates an expression vehicle, wherein the proteoliposome includes an activation specific αᵥβ₃ ligand.

Typical tissues which are exemplary targets for delivery of a therapeutic agent according to the method of the present invention are any tissue in which αᵥβ₃ is expressed and activated, such that delivery presents the agent specifically to the activated αᵥβ₃-containing tissues. These tissues may include, for example neovascular cells, smooth muscle cells, endothelial cells, in particular smooth muscle endothelial cells, arterial cells, osteoclasts, tumor cells, and the like, although the invention need not be so limited. In a preferred embodiment the therapeutic agents are targeted to αᵥβ₃ expressing endothelial cells in the neovasculature of malign tumors.

The agent can be presented by the present methods by any of a variety of means in a therapeutic composition containing the ligand. Typically the agent is operatively linked to the ligand, as by conjugation, chemical linkage or other covalent association, although non-covalent methods may also be utilized which depend upon, for example, specific binding interactions, chemical affinities, and the like.

The invention also contemplates nucleic acid expression vectors for producing a therapeutic fusion protein according to the teachings of the present invention. A therapeutic fusion protein comprises an activated αᵥβ₃ specific ligand operatively linked to a biologically active polypeptide, and is useful to target the biologically active polypeptide to those tissues containing an activated αᵥβ₃.

The activated αᵥβ₃ specific ligand can be any of the ligands described in the present invention. A preferred ligand is the 50 amino acid residue sequence of penton base substituted into PAC1 antibody as described above. Another preferred ligand is the domain of Fab WOW-1 which immunoreacts with activated αᵥβ₃, such as the heavy chain CDR3 domain of WOW-1.

A biologically active polypeptide, discussed hereinabove, can be any polypeptide which imparts a biological function of therapeutic interest to the fusion protein, and therefore the invention need not be so limited. Exemplary polypeptide include the active portion of diphtheria toxin, ricin, peptide hormones, peptide cellular activators, chemokines, cytokines, kinases, and the like biologically active polypeptides.

An expression vector of this invention can be any of a variety of well known constructs suitable for expression of a gene which encodes a fusion protein of this invention, and need not be limited. Exemplary vectors include procaryotic and eukaryotic vectors, particularly retroviral and adenoviral vectors well known in the art for delivery of expressible genes to mammals, particularly humans.

Other uses will be apparent to one skilled in the art in light of the present disclosures. The examples that follow illustrate preferred embodiments of the present invention and are not limiting the description or claims in any way.

### EXAMPLES

### Example 1

Preparation of Soluble αᵥβ₃ Ligands

Recombinant penton base from adenovirus type 2 was baculovirus-expressed in Trichoplusia Tn 5B1-4 insect cells and purified as described previously (Wickham, T. J., Mathias, P., Cheresh, D. A., and Nemerow, G. R. (1993) Cell 73, 309-319). The purified protein migrated as a single ˜325 kDa band on native polyacrylamide gels and an ˜80 kDa band on SDS-polyacrylamide gels. Penton base was conjugated to Alexa-488 to form Alexa-penton base (aPB) according to the manufacturer's instructions (Molecular Probes, Eugene, Oreg.). Purified human fibrinogen was obtained from Enzyme Research Laboratories (South Bend, Ind.) and labeled with FITC (Shattil, S. J., Cunningham, M., and Hoxie, J. A. (1987) Blood. 70, 307-315).

WOW-1 Fab was created by replacing the 19 amino acid H-CDR3 of antibody PAC1 Fab (Abrams, C., Deng, J., Steiner, B., and Shattil, S. J. (1994) J. Biol. Chem. 269, 18781-18788) with the 50 amino acid αᵥ integrin-binding domain from adenovirus type 2 penton base (Mathias, P., Wickham, T., Moore, M., and Nemerow, G. (1994) J Virol 68(10), 6811-4) by splice-overlap PCR using oligonucleotides PB-For (5′-

| | | |
|---|---|---|
| PB-For | (5′-ACACAGCCATATATTACTGTGCCAGAGCGGAAGAGAACTCCAACGCG;, | (SEQ ID NO 1) |
| PB-Rev | (5′-ACTGAGGTTCCTTGACCCCACGCAGCGGGGGCGGCAGCTTCTGC;, | (SEQ ID NO 2) |
| Pac1-For | (5′-GCGCGGGAGATCTCAGGTGCAGCTGAAGCAGTCAGGA; and | (SEQ ID NO 3) |
| Pac1-Rev | (5′-GGCGCATGACCGGTACAATCCCTGGGCACAATTTTCTTG;. | (SEQ ID NO 4) |

The resulting WOW-1 Fd DNA fragment was digested with Bg/II/Agel and cloned into a Drosophila expression vector, pMT/BiP/V5-His B (Invitrogen, Carlsbad, Calif.), which contains the Drosophila metallothionine promoter and BiP secretion signal and places a (His)₆ tag at the C-terminus of Fd. Similarly, PAC1 κ containing Ncol and Agel sites was amplified by PCR with κ-For (5′-GGCGCGGGAGATCTCCATGGGATGTlllGATGACCCMACTCCA; (SEQ ID NO 5) and κ-Rev (5′-GGCGCATGACCGGTACACTCATTCCTGTTGAAGCTCTTG; (SEQ ID NO 6), and cloned into pMT/BiP/V5-His B. Nineteen μg of WOW-1 Fd and PAC1 κ in pMT/BiP/V5-His B were cotransfected with 1 μg of selection vector (pCoHYGRO; Invitrogen) into Drosophila melanogaster S2 cells by calcium phosphate precipitation. Stable S2 cell lines were selected with hygromycin-B and screened for secretion of WOW-1 Fab after a 36-72 h induction with 500 μM CuSO₄.

WOW-1 Fab was purified from 250-1000 ml of serum-free medium by column chromatography on Ni—NTA (Qiagen, Calif.). Typical yields were 2-5 mg/L with a purity of ≧90% as estimated on SDS gels stained with silver or Coomassie Blue. WOW-1 Fab migrated as a single ˜58 kDa band on non-reduced SDS gels and reacted on Western blots with a monoclonal antibody specific for a linear epitope in the integrin-binding domain of penton base (Stewart, P. L., Chiu, C. Y., Huang, S., Muir, T., Zhao, Y., Chait, B., Mathias, P., and Nemerow, G. R. (1997) Embo J 16(6), 1189-98), and with affinity-purified goat anti-mouse κ (Biosource International, Camarillo, Calif.). After reduction, WOW-1 Fab migrated as a ˜33 kDa Fd chain and a ˜25 kDa κ chain. There was no evidence of Fd or κ homodimers. As with PAC1 Fab (Abrams, C., Deng, J., Steiner, B., and Shattil, S. J. (1994) 2J. Biol. Chem. 269, 18781-18788), the relative migration of WOW-1 Fab on a Sephadex G-200 column indicated that it was monomeric and, therefore, monovalent in aqueous solution.

Heavy and Light Chain Sequence of WOW-1 Fab:

| | |
|---|---|
| WOW-1 Fab Heavy chain sequence | |
| CAGGTGCAGCTGAAGCAGTCAGGACCTGGCCTAGTGCAGCCCTCACAGAGCCTGTCCATCACC | (SEQ ID NO 7) |
| TGCACAGTCTCTGGTTTCTCATTAACTAGCTATGGTGTACACTGGGTTCGCCAGTCTCCCGGGA | |
| AGGGTCTGGAGTGGCTGGGAGTGATATGGAGTGGTGGAGGCACAGACTATAATGCAGCTTTCA | |
| TATCCAGACTGAGCATCAGCAAGGACAATTCCAAGAGCCAAGTTTTCTTTAAAATGAACAGTCTG | |
| CAAGCTAATGACACAGCCATATATTACTGTGCCAGAGCGGAAGAGAACTCCAACGCGGCAGCC | |
| GCGGCAATGCAGCCGGTGGAGGACATGAACGATCATGCCATTCGCGGCGACACCTTTGCCACA | |
| CGGGCGGAGGAGAAGCGCGCTGAGGCCGAGGCAGCGGCAGAAGCTGCCGCCCCCGCTGCGT | |
| GGGGTCAAGGAACCTCAGTCACCGTCTCCTCAGCCAAAACGACACCCCCATCTGTCTATCCACT | |
| GGCCCCTGGACTCGCTGCCCAAACTAACTCCATGGTGACCCTGGGATGCCTGGTCAAGGGCTA | |
| TTTCCCTGAGCCAGTGACAGTGACCTGGAACTCTGGATCCCTGTCCAGCGGTGTGCACACCTT | |
| CCCAGCTGTCCTGCAGTCTGACCTCTACAOTCTGAGCAGCTCAGTGACTGTCCCCTCCAGCCC | |
| TCGGCCCAGCGAGACCGTCACCTGCAACGTTGCCCACCCGGCCAGCAGCACCAAGGTGGACA | |
| AGAAANFFGTGCCCAGGGATTGT | |
| WOW-1 Fab Heavy chain amino acid sequence | |
| QVQLKQSGPGLVQPSQSLSITCTVSGFSLTSYGVHVWRQSPGKGLEWLGVIWSGGGTDYNAAFIS | (SEQ ID NO 8) |
| RLSISKDNSKSQVFFKMNSLQANDTAIYYCARAEENSNAAAAAMQPVEDMNDHAIRGDTFATRAEE | |
| KRAEAEAAAEAAAPAAWGQGTSVTVSSAKTTPPSVYPLAPGLAAQTNSMVTLGCLVKGYFPEPVTV | |
| TWNSGSLSSGVHTFPAVLQSDLYTLSSSVTVPSSPRPSETVTCNVAHPASSTKVDKKIVPRDC | |
| WOW-1 Fab Light chain nucleotide sequence | |
| TCTTACATCTATGCGGATCCAGATGTTTTGATGACCCAAACTCCACTCTCCCTGCCTGTCAGTCT | (SEQ ID NO 9) |
| TGGAGATCAAGCCTCCATCCCTTGCAGATCTAGTCAGAGCATTGTACATAGTAATGGAAACACC | |
| TATTTAGAATGGTACCTGCAGAAACCAGGCCAGTCTCCAGCTCCTGATCTACAAAGTTTCCAA | |
| CCGATTTTCTGGGGTCCCAGACAGGTTCAGTGGCAGTGGATCAGGGACAGATTTCACACTCAA | |
| GATCAGCAGAGTGGAGGCTGAGGATCTGGGAGTTTATTACTGCTTTCAAGGTTCACATGTTCCG | |
| TACACGTTCGGAGGGGGGACCAAGCTGGAAATAAAACGGGCTGATGCTGCACCAACTGTATCC | |
| ATCTTCCCACCATCCAGTGAGCAGTTAACATCTGGAGGTGCCTCAGTCGTGTGCTTCTTGAACA | |
| ACTTCTACCCCAAAGACATCAATGTCAAGTGGAAGATTGATGGCAGTGAACGACAAAATGGCGT | |
| CCTGAACAGTTGGACTGATCAGGACAGCAAAGACAGCACCTACAGCATGAGCAGCACCCTCAC | |
| GTTGACCAAGGACGAGTATGAACGACATAACAGCTATACCTGTGAGGCCACTCACAAGACATCA | |
| ACTTCACCCATTGTCAAGAGCTTCAACAGGAATGAGTGT | |
| WOW-1 Fab light chain amino acid sequence | |
| DVLMTQTPLSLPVSLGDQASIPCRSSQSIVHSNGNTYLEWYLQKPGQSPKLLIYKVSNRFSGVPDRF | (SEQ ID NO 10) |
| SGSGSGTDFTLKISRVEAEDLGVYYCFQGSHVPYTFGGGTKLEIKRADAAPTVSIFPPSSEQLTSGG | |
| ASVVCFLNNFYPKDINVKWKIDGSERQNGVLNSWTDQDSKDSTYSMSSTLTLTKDEYERHNSYTCE | |
| ATHKTSTSPIVKSFNRNEC | |

### Example 2

Mammalian Cells and DNA Transfections

cDNAs encoding full-length human αᵥ and β₃ were subcloned into pcDNA3 and pCDM8, respectively, and 2 μg of each were transfected into CHO-K1 cells to obtain transient and stable transfectants as described (O'Toole, T. E., Katagiri, Y., Faull, R. J., Peter, K., Tamura, R., Quaranta, V., Loftus, J. C., Shattil, S. J., and Ginsberg, M. H. (1994) J. Cell Biol. 124, 1047-1059). Stable transfectants surviving antibiotic selection were further screened for high αᵥβ₃ expression by single cell FACS sorting using the αᵥβ₃-specific monoclonal antibody, LM609 (Cheresh, D. A. (1987) Proc. Natl. Acad. Sci. USA. 84, 6471-6475). CHO cells stably expressing wild-type human α_{IIb}β₃ and αᵥβ₃ (D723R) were described previously (O'Toole, T. E., Katagiri, Y., Faull, R. J., Peter, K., Tamura, R., Quaranta, V., Loftus, J. C., Shattil, S. J., and Ginsberg, M. H. (1994) J. Cell Biol. 124, 1047-1059; Hughes, P. E., Diaz-Gonzalez, F., Leong, L., Wu, C. Y., McDonald, J. A., Shattil, S. J., and Ginsberg, M. H. (1996) J. Biol. Chem. 271, 6571-6574). M21-L is a clone of the human melanoma cell line, M21, that lacks the αᵥ subunit (Cheresh, D. A., and Spiro, R. C. (1987) J Biol Chem 262(36), 17703-11). αᵥβ₃-M21-L cells were produced by transient transfection of M21-L with 2 μg each of αᵥ/pcDNA3 and β₃/pCDM8 using Superfect (Qiagen Inc., Chatsworth, Calif.). CS-1 is a hamster melanoma cell line that does not express αᵥβ₃ or αᵥβ₅ because it does not synthesize the β₃ or β₅ subunits. αᵥβ₃-CS-1 cells stably expressing hamster αᵥ and human β₃ were obtained by transfection of CS-1 cells with human β₃ (Filardo, E. J., Brooks, P. C., Deming, S. L., Damsky, C., and Cheresh, D. A. (1995) J. Cell Biol. 130, 441-450). JY is an immortalized human B-lymphoblastoid cell line that expresses αᵥβ₃ but not αᵥβ₅ (Stupack, D. G., Shen, C., and Wilkins, J. A. (1992) Exp. Cell Res. 203, 443-448; Rothlein, R., and Springer, T. A. (1986) J Exp Med 163(5), 1132-49).

### Example 3

Analysis of Cell Surface Integrin Expression

Cells were suspended in an “incubation buffer” (137 mM NaCl, 2.7 mM KCl, 3.3 mM NaH₂PO₄, 3.8 mM HEPES, 1 mM MgCl₂, 5.5 mM glucose, and 1 mg/ml bovine serum albumin, pH 7.4) and incubated for 30 min on ice with a monoclonal antibody (10 μg/ml) specific for either αᵥβ₃ (LM609), α_{IIb}β₃ (D57) (O'Toole, T. E., Katagiri, Y., Faull, R. J., Peter, K., Tamura, R., Quaranta, V., Loftus, J. C., Shattil, S. J., and Ginsberg, M. H. (1994) J. Cell Biol. 124, 1047-1059) or αᵥβ₅ (P1F6) (Lin, E. C. K., Ratnikov, B. I., Tsai, P. M., Carron, C. P., Myers, D. M., Barbas, C. F., III, and Smith, J. W. (1997) J. Biol. Chem. 272, 23912-23920). After washing, the cells were incubated another 30 min on ice with FITC-conjugated goat anti-mouse IgG (H+L chain-specific; Biosource), washed again, and analyzed on a FACSCalibur flow cytometer (Becton Dickinson, Mountain View, Calif.) (Hato, T., Pampori, N., and Shattil, S. J. (1998) J. Cell Biol. 141(7), 1685-1695). As a negative control, samples were incubated with the secondary antibody alone.

### Example 4

Ligand Binding Assays

Binding of aPB, WOW-1 Fab and FITC-fibrinogen to cells was assessed by flow cytometry. Typically, cells were cultured overnight in low serum medium (e.g., 0.5% fetal bovine serum), resuspended in incubation buffer at 1-1.5×10⁷ cells/ml, and 4-6×10⁵ cells were incubated with one of these ligands for 30 min at room temperature in a final volume of 50 μl. As indicated, some samples were also incubated in the presence of one or more of the following reagents: antibody AP5 ascites (1:50) to activate β₃ integrins (Pelletier, A. J., Kunicki, T., Ruggeri, Z. M., and Quaranta, V. (1995) J. Biol. Chem. 270, 18133-18140), 0.25 mM MnCl₂to activate integrins (Bazzoni, G., and Hemler, M. E. (1998) Trends Biochem. Sci. 23, 30-34), 2 mM RGDS or 5 mM EDTA to specifically block ligand binding to integrins, 50 μM cRGDfV, a selective αᵥ integrin antagonist (Peninsula Laboratories, Inc., Belmont, Calif.), 5 μM Integrilin, a selective α_{IIb}β₃ antagonist (Scarborough, R. M., Naughton, M. A., Teng, W., Rose, J. W., Phillips, D. R., Nannizzi, L., Arfsten, A., Campbell, A. M., and Charo, I. F. (1993) J. Biol. Chem. 268, 1066-1073) or 100 μg/ml of the function-blocking antibodies, LM609 or P1F6. In some experiments, ligand binding and αᵥβ₃ expression were measured simultaneously by incubation of cells with ligands in the presence of biotin-SSA6 (7 μg/ml), a non-function-blocking anti-β₃ monoclonal antibody (Abrams, C., Deng, J., Steiner, B., and Shattil, S. J. (1994) J. Biol. Chem. 269, 18781-18788). After 30 min at room temperature, cells were washed and incubated with phycoerythrin-streptavidin (1:25 final dilution; Molecular Probes) for 20 min on ice. In the case of WOW-1 Fab, an Alexa-conjugated anti-(His)₆ monoclonal antibody (Accurate Chemical and Scientific Corp., Westbury, N.Y.) was added at this stage (50 μg/ml). Cells were washed and resuspended in 0.5 ml incubation buffer containing 2 μg/ml propidium iodide (Sigma, St. Louis, Mo.). Ligand binding (FL1 channel) was analyzed immediately on the gated subset of live cells (propidium iodide-negative, FL3) that was strongly positive for αᵥβ₃ expression (FL2). Binding isotherms were subjected to non-linear, least squares regression analysis using an equation for one-site binding (Prism 2.0 software; GraphPad Software, San Diego, Calif.). Two-tailed P values for paired samples were obtained by Student's t test.

To examine the effects of overexpression of isolated integrin cytoplasmic tails on ligand binding to αᵥβ_{3, α}ᵥβ₃-CS-1 cells were transfected with a mammalian expression plasmid encoding either Tac-β₁, Tac-β₃ or Tac-α₅, using Fugene-6 transfection reagent (Boehringer Mannheim, Indianapolis, Ind.) (LaFlamme, S. E., Thomas, L. A., Yamada, S. S., and Yamada, K. M. (1994) J. Cell Biol. 126, 1287-1298; Chen, Y. -P., O'Toole, T. E., Shipley, T., Forsyth, J., LaFlamme, S. E., Yamada, K. M., Shattil, S. J., and Ginsberg, M. H. (1994) J. Biol. Chem. 269, 18307-18310). Forty-eight hours after transfection, cells were suspended in incubation buffer at 1.5×10⁶/ml and incubated for 30 min at room temperature with 150 nM aPB or 425 nM WOW-1 Fab in the presence or absence of 5 mM EDTA. After washing, cells were incubated for an additional 30 min on ice with 2.5 μg/ml of biotinylated anti-Tac monoclonal antibody (7G7B6), followed by incubation with phycoerythrin-conjugated anti-mouse IgG, and (when WOW-1 Fab was present) 50 μg/ml of Alexa-anti-(His)₆. Ligand binding was analyzed on the gated subset of live cells strongly positive for Tac expression. In parallel tubes, cells were co-stained with SSA6 and anti-Tac antibody to quantitate αᵥβ₃ expression in the Tac-positive cells.

Binding of WOW-1 Fab to purified αᵥβ₃ receptors from human placenta and α_{IIb}β₃ from human platelets was measured by ELISA in the presence of 50 μM CaCl₂, MgCl₂ and MnCl₂. Non-specific binding was determined in the presence of 2 mM RGDS (Abrams, C., Deng, J., Steiner, B., and Shattil, S. J. (1994) J. Biol. Chem. 269, 18781-18788).

### Example 5

Cell Adhesion Assays

Immulon-2 microtiter wells (Dynex Laboratories, Chantilly, Va.) were coated with unlabeled penton base (1-100 ng/well) overnight at 4° C., followed by blocking with 20 mg/ml BSA. CHO cells stably expressing αᵥβ₃ were labeled with BCECF-AM (Molecular Probes, Eugene, Oreg.), and cell adhesion to the immobilized penton base was quantitated by cytofluorimetry at 485/530 nm (Hato, T., Pampori, N., and Shattil, S. J. (1998) J. Cell Biol. 141(7), 1685-1695).

### Example 6

Adenovirus-Mediated Gene Delivery

CS-1 and αᵥβ₃-CS-1 cells (10⁵ cells) were suspended for 5 min at room temperature in 100 μl of incubation buffer. In some cases, 2.5 mM MnCl₂ was also present to induce maximal integrin activation. Then replication-deficient adenovirus type 5 encoding green fluorescent protein (GFP) was added to the cell suspension at a multiplicity of infection (m.o.i) of 50 or 500 (Huang, S., Stupack, D., Mathias, P., Wang, Y., and Nemerow, G. (1997) Proc Natl Acad Sci USA 94(15), 8156-61). After 1 h at 37° C., virus not internalized was digested by incubation of the cells with 0.03% trypsin/0.35 mM EDTA for 5 min at 37° C. After 72 h, GFP expression was quantitated by flow cytometry.

### Example 7

Interaction of a Novel Monovalent Ligand with Interin αᵥβ₃

In order to document and study the significance of affinity modulation of αᵥβ₃, a monovalent reporter ligand was developed analogous to the activation-dependent anti-α_{IIb}β₃ antibody, PAC1 Fab. Preliminary binding studies were conducted with the new antibody, designated WOW-1 Fab, using purified integrins in the presence of 50 μM MnCl₂, which activates integrins by a direct effect on the extracellular domain (Bazzoni, G., and Hemler, M. E. (1998) Trends Biochem. Sci. 23, 30-34). WOW-1 Fab bound to purified αᵥβ₃ and to a lesser extent to purified αᵥβ₅. Binding was half-maximal at 40 nM Fab and was blocked by >95% by 2 mM RGDS or 5 mM EDTA. In contrast, there was no detectable binding of WOW-1 Fab to purified α_{IIb}β₃ at antibody concentrations as high as 2 μM, even though the parent antibody, PAC1 Fab, bound half-maximally to α_{IIb}β₃ at 50 nM. These results indicate that the re-engineering of PAC1 Fab has converted it from an activation-dependent α_{IIb}β₃ antibody into an antibody that reacts with activated αᵥβ₃. To determine if WOW-1 Fab reacted preferentially with activated αᵥβ₃ in cells, Fab binding was compared with that of multivalent penton base using CHO cells stably-transfected with human αᵥβ₃ (αᵥβ₃-CHO cells). Flow cytometric analysis showed that the surface of these cells expressed large amounts of αᵥβ₃, modest amounts of αᵥβ₅ and no detectable α_{IIb}β₃ (FIG. 1A). When Alexa-penton base (aPB) or WOW-1 Fab was incubated with the cells over a range of ligand concentrations (5-1000 nM) and for various periods of time at room temperature, specific ligand binding, defined as that inhibitable by 2 mM RGDS or 5 mM EDTA, reached a steady state by 30 min, and non-specific binding accounted for ≦15% of total binding. Therefore, all subsequent binding studies were carried out under these conditions. aPB and WOW-1 Fab bound specifically but at low levels to unstimulated αᵥβ₃-CHO cells. However, direct in activation of αᵥβ₃ by anti-β₃ antibody AP5 caused a significant increase in the binding of both ligands (P<0.01) (FIG. 1B).

To assess the selectivity of these ligands for αᵥβ₃ in this system, the effect of various function-blocking compounds was studied. Binding of aPB and WOW-1 Fab in the presence of antibody AP5 was inhibited ≧85% by 2 mM RGDS or 50 μM cRGDfV, a cyclic peptide selective for αᵥ integrins (FIG. 2) (Brooks, P. C., Montgomery, A. M. P., Rosenfeld, M., Reisfeld, R. A., Hu, T., Klier, G., and Cheresh, D. A. (1994) Cell 79,1157-1164). On the other hand, a cyclic peptide selective for α_{IIb}β₃ (Integrilin) inhibited ligand binding by less than 20%, even at a concentration (1 μM) 100-fold higher than that necessary to prevent fibrinogen or PAC1 binding to platelet α_{IIb}β₃ (Scarborough, R. M., Naughton, M. A., Teng, W., Rose, J. W., Phillips, D. R., Nannizzi, L., Arfsten, A., Campbell, A. M., and Charo, I. F. (1993) J. Biol. Chem. 268, 1066-1073). Furthermore, the αᵥβ₃function-blocking antibody LM609 (100 μg/ml) inhibited ligand binding by more than 70%, while the αᵥβ₅ blocking antibody P1F6 had no such effect. In addition, neither aPB nor WOW-1 Fab bound detectably to resting or thrombin-stimulated human platelets, which express >50,000 α_{IIb}β₃ receptors but less than 500 αᵥβ₃ receptors per cell (Coller, B. S., Cheresh, D. A., Asch, E., and Seligsohn, U. (1991) Blood 77, 75-83). Collectively, these results indicate that a monovalent ligand, WOW-1 Fab, and a multivalent ligand, aPB, are sensitive to the activation state of αᵥβ₃ and they do not recognize α_{IIb}β₃. Thus, WOW-1 Fab is a suitable reporter for changes in αᵥβ₃ affinity. Since WOW-1 Fab (and aPB) also recognize αᵥβ₅, particular efforts were made in the experiments that follow to utilize cells that express αᵥβ₃ but little or no αᵥβ₅.

### Example 8

The Affinity of αᵥβ₃ can be Regulated by Inside-Out Signals

To determine if αᵥβ₃ is susceptible to affinity modulation by inside-out signals, the binding of WOW-1 Fab to JY B-lymphoblasts was studied. These cells were selected because they express αᵥβ₃ but not αᵥβ₅ and they adhere rapidly to vitronectin in response activation of protein kinase C by phorbol myristate acetate (Stupack, D. G., Shen, C., and Wilkins, J. A. (1992) Exp. Cell Res. 203, 443-448; Rothlein, R., and Springer, T. A. (1986) J Exp Med 163(5), 1132-49). Incubation of JY cells for 15 min with 100 nM phorbol myristate acetate caused a significant increase in specific binding of aPB (2.7±0.2-fold increase; P<0.05), consistent with an increase in αᵥβ₃ affinity and/or avidity. Furthermore, phorbol myristate acetate caused a 2.4±0.1-fold increase in the binding of WOW-1 Fab (P<0.05). Neither response was increased further by activating antibody AP5 (FIG. 3A). Phorbol myristate acetate did not increase the surface expression of αᵥβ₃, as measured by antibody LM609. To determine whether the changes in WOW-1 Fab binding reflected changes in αᵥβ₃ affinity, ligand binding was analyzed over a range of antibody concentrations. Unstimulated JY cells exhibited a very low affinity for WOW-1 Fab (apparent Kd=2,600±700 nM; SEM) and a value for maximal binding of 24.8±4.1 arbitrary fluorescence units (FIG. 3B). In marked contrast, JY cells stimulated with phorbol myristate acetate exhibited a >30-fold increase in binding affinity (apparent Kd=80±18 nM) with no change in maximal binding (23.5±1.1 units). This effect was prevented if the cells were first depleted of metabolic energy by a 30 min preincubation with 0.2% sodium azide and 4 mg/ml 2-deoxy-d-glucose. These results establish that energy-dependent inside-out signals can regulate the ligand binding affinity of αᵥβ₃.

### Example 9

Determinants of αᵥβ₃ Activation State

Experiments were performed to identify factors that influence αᵥβ₃ affinity using readily transfectable cell lines that stably express human αᵥβ₃. αᵥβ₃ on vascular cells may encounter multiple ligands simultaneously during the process of wound healing. Therefore, it was assessed whether the affinity/avidity of αᵥβ₃ differed for various ligands. Equilibrium binding of aPB, WOW-1 Fab, and the adhesive ligand, fibrinogen, was compared in αᵥβ₃-CHO cells. As summarized in Table 1, each ligand bound specifically to approximately the same total number of receptors in unstimulated αᵥβ₃-CHO cells. However, the affinity/avidity of αᵥβ₃ for fibrinogen was approximately 15-fold lower than that for aPB, despite the fact that both ligands are multivalent and similar in molecular mass. Activation of αᵥβ₃ with antibody AP5 increased the binding affinity/avidity for both ligands but it had no effect on maximal binding (Table 1). On the other hand, despite the differences in valency between aPB and WOW-1 Fab, their binding constants were similar. Overall, these results show that αᵥβ₃ can interact differentially with macromolecular ligands and that the affinity state of the receptor is one determinant of such interactions.

TABLE 1
Binding of different ligands to αᵥβ₃ expressed in CHO cells*

| | | | Activating | |
|---|---|---|---|---|
| | No Treatment | | antibody AP5 | |
| | Apparent Kd* | Bmax | Apparent Kd | Bmax |
| Ligand | (nM) | (units) | (nM) | (units) |
| WOW-1 Fab | 514 ± 71 | 62 ± 3 | 119 ± 12 | 65 ± 2 |
| Penton Base | 550 ± 53 | 80 ± 4 | 160 ± 31 | 69 ± 5 |
| Fibrinogen | 9,200 ± 6,500 | 126 ± 74 | 566 ± 110 | 77 ± 6 |

*Ligand binding was determined by flow cytometry and binding isotherms were analyzed as described in Experimental Procedures and in the legend to FIG. 3. Data represent the combined results of three independent experiments with each ligand. Maximum binding (Bmax) was expressed in arbitrary fluorescence units. Goodness of fit (R²) values ranged from 0.93-1.00.

In circulating platelets, the “basal” activation state of α_{IIb}β₃ must remain low to prevent thrombosis. However, this requirement may not pertain to all cells that express αᵥβ₃. Therefore, ligand binding was studied simultaneously in αᵥβ₃-CHO cells and in two unrelated melanoma cell lines, αᵥβ₃-M21-L and αᵥβ₃-CS-1, to assess cell type-specific variations in basal activation state of αᵥβ₃. In order to control for minor variations in αᵥβ₃ expression between the cell lines, ligand binding was expressed on a “per receptor” basis using anti-β₃ antibody SSA6 to quantitate receptor expression. Unstimulated αᵥβ₃-M21-L cells bound significantly more aPB than did αᵥβ₃-CHO cells (P<0.01). This difference was maintained even after further activation of αᵥβ₃ with antibody AP5 (P<0.05) (FIG. 4). Similar results were obtained with αᵥ62 ₃-CS-1 cells instead of αᵥβ₃-M21-L cells, and with WOW-1 Fab instead of aPB. Taken together with the marked differences observed in the binding of WOW-1 Fab to unstimulated JY lymphoblasts and αᵥβ₃-CHO cells (FIG. 3B and Table 1), these results indicate that the basal activation state of αᵥβ₃ varies with the cell type.

Integrin cytoplasmic tails have been implicated in affinity/avidity modulation of several integrins (Hemler, M. E. (1998) Current Opinion in Cell Biology 10, 578-585), but there is no direct information about their role in regulating ligand binding to αᵥβ₃. Certain point mutations or truncations of the β₃ cytoplasmic tail, such as β₃ (D723R), result in constitutive activation of α_{IIb}β₃ in CHO cells (O'Toole, T. E., Katagiri, Y., Faull, R. J., Peter, K., Tamura, R., Quaranta, V., Loftus, J. C., Shattil, S. J., and Ginsberg, M. H. (1994) J. Cell Biol. 124, 1047-1059; Hughes, P. E., Diaz-Gonzalez, F., Leong, L., Wu, C. Y., McDonald, J. A., Shattil, S. J., and Ginsberg, M. H. (1996) J. Biol. Chem. 271, 6571-6574). To determine whether αᵥβ₃ is affected by such a modification, ligand binding to αᵥβ₃ (D723R) was assessed. This mutant was stably-expressed in CHO cells to approximately the same level as wild-type αᵥβ₃ (FIG. 5A). However, unstimulated αᵥβ₃ (D723R)-CHO cells bound significantly more aPB than unstimulated αᵥβ₃-CHO cells (P<0.01), equivalent to the amount of aPB bound to αᵥβ₃-CHO cells treated with AP5 (FIG. 5B). A second αᵥβ₃ (D723R) clone gave the same results, and similar results were obtained using WOW-1 Fab instead of aPB. Thus, a structural change in the β₃ cytoplasmic tail can be propagated to the extracellular domains of αᵥβ₃ to influence ligand binding affinity.

The activation state of certain integrins, such as α_{IIb}β₃ and α₅β₁, can be suppressed in a dominant-inhibitory fashion by overexpression of isolated β₃ or β₁ cytoplasmic tails, but not by α₅ tails (LaFlamme, S. E., Thomas, L. A., Yamada, S. S., and Yamada, K. M. (1994) J. Cell Biol. 126, 1287-1298; Chen, Y. -P., O'Toole, T. E., Shipley, T., Forsyth, J., LaFlamme, S. E., Yamada, K. M., Shattil,2 S. J., and Ginsberg, M. H. (1994) J. Biol. Chem. 269, 18307-18310; Kashiwagi, H., Schwartz, M. A., Eigenthaler, M. A., Davis, K. A., Ginsberg, M. H., and Shattil, S. J. (1997) J. Cell. Biol. 137, 1433-1443). To determine if αᵥβ₃ is subject to this type of suppression, αᵥβ₃-CS-1 cells were transiently-transfected with chimeric constructs consisting of the β₃, β₁, or α₅, cytoplasmic tails fused at their N-termini to the extracellular and transmembrane domains of the Tac subunit of the IL2 receptor, which was used to target the tails to the vicinity of the plasma membrane. Despite similar levels of expression of the chimeras, Tac-β₃ and Tac-β₁ caused a significant reduction in specific binding of aPB and WOW-1 Fab when compared to Tac-α₅ (P<0.01) (FIG. 6A, B). In contrast, none of these tail chimeras affected surface expression of αᵥβ₃ (FIG. 6C). Since the isolated β tails may bind proteins that normally interact with integrins (LaFlamme, S. E., Thomas, L. A., Yamada, S. S., and Yamada, K. M. (1994) J. Cell Biol. 126, 1287-1298), these results suggest that αᵥβ₃ may be regulated by direct interactions with intracellular proteins.

### Example 10

Functional Consequences of Affinity Modulation of αᵥβ₃

In order to determine whether changes in receptor affinity affect the adhesive function of αᵥβ₃, the adhesion of αᵥβ₃-CHO cells to immobilized penton base was quantitated. Adhesion was dependent on the coating concentration of penton base and was half-maximal at 30-40 ng/well (FIG. 7). Activation of αᵥβ₃ by AP5 led to a 7-fold leftward shift in the dose-response curve such that half-maximal adhesion now occurred at approximately 5 ng of penton base/well. Treatment of the cells with 1 mM MnCl₂ caused an even further shift in the dose-response curve, either because it induced a more profound effect on αᵥβ₃ or it activated additional αᵥ integrins (FIG. 7). Analysis of adherent cells by light microscopy showed that they had become fully-spread by 90 min. Thus, affinity modulation of αᵥβ₃ promotes both cell adhesion and post-ligand binding responses, such as cell spreading.

Adenoviruses utilize αᵥ integrins to enter cells and are a common gene delivery vector. Therefore, we tested whether changes in αᵥβ₃ affinity could influence adenovirus-mediated gene transfer. Recombinant adenovirus containing cDNA encoding GFP was incubated with CS-1 melanoma cells at an m.o.i. of 50 and 500, and subsequent cellular expression of GFP was taken as a marker for infection and gene transfer. CS-1 cells were chosen because they do not express αᵥβ₅, thus potentially restricting adenovirus internalization through stably expressed αᵥβ₃. When parental cells without αᵥβ₃ were incubated with virus for 60 min and monitored for infection 72 hours later, they exhibited a relatively low level of GFP expression. Unstimulated αᵥβ₃-CS-1 cells exhibited a higher level of GFP expression, particularly at the higher m.o.i. (FIG. 8A). However, if incubation of αᵥβ₃-CS-1 cells with virus was carried out in the presence of 2.5 mM MnCl₂ to activate αᵥβ₃, the cells subsequently exhibited a much greater increase in GFP expression at the lower m.o.i (P<0.01) (FIGS. 8A and B, first three bars on the left). MnCl₂ had no effect on GFP expression in the parental CS-1 cells. Enhanced GFP expression in cells containing activated αᵥβ₃ was blocked if the cells were preincubated with an excess of WOW-1 Fab (1.7 μM) before the addition of virus (FIG. 8B, 4^{th} bar from the left). Thus, adenovirus-mediated gene transfer is directy affected by affinity modulation of αᵥβ₃.

Abbreviations used: RGD, single letter code for amino acids Arg, Gly and Asp; aPB, Alexa-penton base; GFP, green fluorescent protein; m.o.i, multiplicity of infection.

1. A method for detecting the presence of activated vitronectin receptor αᵥβ₃ in a tissue comprising:
(a) admixing a ligand which binds activated vitronectin receptor αᵥβ₃ with a tissue containing αᵥβ₃;
(b) maintaining said admixture under conditions sufficient for said ligand to bind said αᵥβ₃ and form a ligand-αᵥβ₃ complex;
(c) determining the presence of said ligand-αᵥβ₃ complex, and thereby the presence of said activated αᵥβ₃ in said tissue.

2. The method of claim 1 wherein said ligand is selected from the group consisting of adenovirus-2 penton base and an antibody that immunoreacts with activated αᵥβ₃.

3. The method of claim 2 wherein said ligand is the Fab antibody WOW-1.

4. The method of any of claims 1 to 3 wherein said ligand comprises a label and said determining of step (c) comprises detecting the presence of said label in said complex.

5. The method of any of claims 1 to 4 wherein said tissue comprises neovascular cells, smooth muscle endothelial cells, arterial cells, osteoclasts and tumor cells.

6. A method for delivery of an agent in a therapeutic composition to a tissue containing activated vitronectin receptor αᵥβ₃ comprising:
(a) contacting a tissue containing said αᵥβ₃ with a therapeutic composition comprising a ligand that binds to activated αᵥβ₃, wherein said ligand is operatively linked to an agent and said agent has a therapeutic activity;
(b) maintaining said therapeutic composition in contact with said tissue under conditions sufficient for said ligand to bind to said activated αᵥβ₃ and thereby deliver said agent to said tissue.

7. The method of claim 6 wherein said contacting is conducted between said tissue and said therapeutic composition ex vivo.

8. The method of claim 6 wherein said contacting is conducted between said tissue and said therapeutic composition in vivo.

9. The method of any of claims 6 to 8 wherein said ligand is selected from the group consisting of adenovirus-2 penton base, a penton base fragment that binds activated αᵥβ₃, and an antibody that immunoreacts with activated αᵥβ₃.

10. The method of claim 9 wherein said ligand is the Fab antibody WOW-1.

11. The method of any of claims 6 to 10 wherein said agent is a biologically active compound.

12. The method of claim 11 wherein said agent is a nucleic acid selected from the group consisting of a gene, an antisense nucleic acid and a catalytic nucleic acid.

13. The method of any of claims 6 to 12 wherein said tissue comprises neovascular cells, smooth muscle endothelial cells, arterial cells, osteoclasts and tumor cells.

14. An isolated antibody molecule which immunoreacts with activated vitronectin receptor αᵥβ₃.

15. The antibody of claim 14 wherein said antibody is a Fab, Fd, Fv, scFv fragment or intact immunoglobulin molecule.

16. The antibody of any of claims 14 or 15 wherein said antibody comprises a penton base fragment that binds activated αᵥβ₃.

17. The antibody of any of claims 14 to 16 wherein said antibody comprises a single αᵥ integrin-binding domain from a multivalent adenovirus penton base

18. The antibody of any of claims 14 to 17 wherein said antibody comprises an amino acid residue sequence shown Sequence Id. No. 8 or Sequence Id. No. 10.

19. The antibody of claim 18 wherein said antibody is Fab WOW-1.

20. A nucleic acid expression vector comprising an expression cassette capable of expressing a nucleotide sequence which encodes a fusion protein, said fusion protein comprising an activated αᵥβ₃ specific ligand operatively linked to a biologically active agent.

21. The vector of claim 20 wherein said ligand is selected from the group consisting of adenovirus-2 penton base, a penton base fragment that binds activated αᵥβ₃ and an antibody that immunoreacts with activated αᵥβ₃.

22. The vector of claim 21 wherein said ligand is the αᵥ integrin-binding domain from adenovirus type 2 penton base.

23. The vector of claim 21 wherein said ligand comprises the CDR3 domain of Fab WOW-1.

24. The vector of claim 21 wherein said ligand comprises the activated αᵥβ₃ binding domain of Fab WOW-1.
